(19) United States
(12) Patent Application Publication
(10) Pub. No.: US 2023/0076664 A1
(43) Pub. Date: Mar. 9, 2023

(54) MULTIFUNCTIONAL FACE MASK / HELMET FACE COVERING WITH UNIVERSAL COOL FILTER ICE PACK INSERTS

(71) Applicant: Dolhman Brown, SR., Harvey, LA (US)

(72) Inventor: Dolhman Brown, SR., Harvey, LA (US)

(21) Appl. No.: 17/468,451

(22) Filed: Sep. 7, 2021

(51) Int. Cl. A61F 7/02; A62B 18/02

(52) U.S. Cl. CPC A61F 7/02; A62B 18/02; A61F 2007/0003

(57) ABSTRACT

A multifunctional face mask assembly apparatus to help to prevent or reduce heat-related illness for individuals working in high temperature environments. Fastened to the face, head and neck, to provide a cool ambient microclimate and/or aid in lowering skin temperature around the face, head and neck to aid in cooling the regulation of cerebral blood flow by means of the Hypothalamus which is responsible for regulating body temperature. The multifunctional face mask assembly use ice cooling technology to help lower temperatures when experiencing hot flashes, aid in reducing the onset of a fever, aid in improving performance by means of pre-cooling, intermittent cooling, per/mid-cooling or post-cooling during exertional sports, fitness and/or exercises, use in Cooling Centers to lower temperature around the face, head and neck. The apparatus is optionally housed, secured or attached within the nose cup cavity opening of an elastomeric half facepiece reusable respirator, full facepiece respirator, resistance training mask, dust particle mask or face mask. The apparatus is optionally use to housed, secured or attached to handheld/portable Nebulizer mask, elastomeric half facepiece reusable respirator, resistance training mask, dust particle mask or face mask nose cup. The apparatus is fashioned, designed, shaped or configured for use as a Helmet Face Covering for (e.g., professional, college, schools, recreation, entertainment, leisure or, outdoor recreational activities).

This application claims priority benefit of provisional application Ser. No. 63/077,954 filed on Sep. 14, 2020, incorporated herein by reference as if set forth in full below.

### BACKGROUND OF THE INVENTION

### I. Field of the Invention

The present invention pertains to a multifunctional face mask assembly embodiment comprising a Filtrator™ nose cup accessory, cartridge filter adapter, a filter cover with air vents, a filter, a universal cool release vent harness sleeve/strap accessory, a removable universal cool filter ice pack insert accessory, a universal cool filter ice pack insert pouch accessory used to provide cool ambient microclimate around the face, to aid in lowering skin temperature around the face, head and neck. The removable universal cool filter ice pack insert accessory is optionally housed, secured or attached within the cavity opening of a elastomeric half facepiece reusable respirator, resistance training mask, dust particle mask or face mask. The universal cool release vent harness sleeve/strap accessory is optionally used to house or secure a elastomeric half facepiece reusable respirator, resistance training mask, dust particle mask or face mask. The universal cool release vent harness sleeve/strap accessory is optionally used to aid in improving performance by means of pre-cooling, intermittent cooling, per/mid-cooling or post-cooling during exertional sports, fitness and/or exercises.

### II. Background of the Invention

Before COVID-19 was characterized as a pandemic throughout the world, traditional, conventional or modern respirators, surgical masks, elevation/resistance training mask and/or face mask/coverings was generally worn by healthcare professional or para-professional individuals (e.g., physicians, nurses, emergency medical service personnel, nursing assistants, technicians, therapists, phlebotomists, pharmacists, students and trainees involved in patient care, industrial workers, agriculture, farmworkers, spray painters, welders, miners, construction workers, first responders, fireman, law enforcement officers and/or for resistance fitness training). Climate change and increasing heat waves are beginning to have more effects on people experiencing heat-related illness throughout the world. This includes those who wear traditional, conventional or modern respirators, surgical masks, elevation/resistance training mask and/or face mask/coverings. Because there is an increase in heat-related illness injuries, professionals, industrial workers, para-professionals, sports, recreational, home and/or leisure settings are at risk. Although there are recommendations to help prevent heat-related illness, they do not provide a multifunctional face mask assembly embodiment comprising a universal cool release vent harness sleeve/strap accessory that houses or secure a removable universal cool filter ice pack insert accessory to fastened to the face, head and neck, to provide a cool ambient microclimate around the face and/or aid in cooling the regulation of cerebral blood flow by means of the Hypothalamus which is responsible for regulating body temperature.

Generally, there are several types of traditional face mask being used during the COVID-19 pandemic. These include but are not limited to, traditional, conventional or modern respirators, surgical masks, elevation/resistance training mask and/or face mask/coverings. Respirators are also called Filtering facepiece respirators (FFR), which are sometimes called disposable respirators are called by different names around the world (e.g., N95 (United States), FFP2 (Europe), KN95 (China), P2 (Australia/New Zealand), Korea 1st class (Korea) and DS2 (Japan). There are several other types of respirators that are not generally used by the public such as Respirators with exhalation valve, Half-Face Respirators, Full-Face Respirators, Respirator with Cartridges. Some of these masks are made and produced by some of the following Companies (e.g., 3M Company, Avon Protection Systems, Inc., Honeywell International, Inc., MSA Safety Inc., Drägerwerk AG & Co. KG, KOWA, Uvex, Halyard Healthcare, McKesson, Molnlycke Health, Hakugen, Shanghai Dasheng, Sinotextiles, Irema and/or Winner Medical).

Some traditional, conventional or modern respirators, surgical masks, elevation/resistance training mask and/or face mask/coverings that the general public wear, are called or described as Elevation/Resistance Training Mask or Olson masks, Face or Neck Gaiters, Fu Face mask, flat-fold tie-on, duckbill with or without shield, cone shaped, reusable mask, Air Filtration Filters (PM 2.5), bandanna, flat-fold with shields, training mask, 3D face mask, Platypus, PM2.5 face mask with or without meltblown filters and/or Filti face mask, just to name a few. The material of these traditional, conventional or modern face mask/coverings can be made from any of the following materials that is non-toxic or harmful to an individual's skin. The materials may comprise of Silicone, Cotton, Jersey Box Lining Fabric, Polyester Sandwich Spacer Mesh, Polyester Warp Knitting Sandwich Mesh, Chamois, Interfacing, foam, Felt, Halyard Health Sequential Sterilization Fabrics, microfiber material, antimicrobial fabrics, spun bond nonwoven fabric, polyester (PES), nanosilver technology, nanofabric, Heat-Activated Thermoplastics that includes ethylene vinyl acetate (EVA), neoprene, polypropylene spun bond nonwoven, breathable nylon mesh, polyolefin materials, poly-nylon and spandex and/or N95-rated material. Some these traditional, conventional or modern face mask/coverings may comprise a pocket flap used to house, secure, store hold or conceal a filter. This filter is used to aid in preventing particles from entering within the face mask, or to allow one's breath to be released through the conventional respirator mask.

However, traditional, conventional or modern respirators, surgical masks, elevation/resistance training mask and/or face mask/coverings do not provide a constant cool ambient microclimate to aid in maintaining lower humidity temperatures within the face mask. Wearing a face mask for a long period of time can increase heat and humidity levels above 90 degrees within the microclimate of traditional, conventional or modern respirators, surgical masks, elevation/resistance training mask and/or face mask/coverings. Increase heat and humidity levels within the microclimate of traditional, conventional or modern respirators, surgical masks, elevation/resistance training mask and/or face mask/coverings can cause the individual to become very uncomfortable in so many ways.

Furthermore, traditional, conventional or modern respirators, surgical masks, elevation/resistance training mask and/or face mask/coverings do not provide a constant cool ambient microclimate by lowering the ambient thermal environment being worn by to those individuals performing certain exercises. Research accordingly, “Subjective and physiological responses were obtained from six subjects wearing a ventilated face mask while exercising (3.8 met) for 15 min on a bicycle ergometer. Different combinations of ambient air temperatures (7 degrees, 16 degrees, 25 degrees C.) and mask air temperatures (22 degrees, 27 degrees, 33 degrees C.) were studied together with two different air humidifies inside the mask (61% and 86% RH). Control experiments were performed without the mask at the same ambient temperatures. Skin temperatures, heart rates and skin wetness were monitored during exercise. The subject's acceptance of the mask and thermal environment, thermal sensation, sensations of discomfort, sweating and skin wettedness, and their judgment of the work of breathing were assessed at the end of the 15 min exercise period. The acceptance of both the ambient thermal environment and of the thermal microclimate in the mask primarily was determined by the ambient air temperature, but it was influenced by the air temperature and humidity inside the mask. At ambient temperatures of 7 degrees C. and 25 degrees C., the acceptance of the thermal work conditions decreased. In the warm environment a mask air temperature less than or equal to 27 degrees C. was 100% acceptable and increased the acceptance of thermal environment. In the cool environment, a mask air temperature greater than or equal to 27 degrees C. was 100% acceptable. The humidity content of the mask air was only important when the mask air was warm. Warm humid air significantly decreased acceptance of the mask conditions.” (R Nielsen, et al., “The Effect of Temperature and Humidity Levels in a Protective Mask on User Acceptability During Exercise.” 1987 July; 48(7):639-45; each of which is incorporated herein by reference in its entirety).

According to an article written by CDC/NIOSH, “Limiting Heat Burden While Wearing Personal Protective Equipment (PPE)”. The question is presented, “Can wearing personal protective equipment (PPE) increase the risk for heat-related illnesses?” “Yes, wearing PPE and certain clothing ensembles can often increase your risk for heat-related illnesses. PPE (e.g., waterproof aprons, surgical gowns, surgical caps, respirators, face shields, boots, and gloves).” Furthermore, CDC/NIOSH pointed out and gave recommendations on “Using wearable personal cooling systems could reduce the time required to lower core body temperature. For example, during rest breaks: Remove PPE and clothing ensembles. While rehydrating, apply active (e.g., Cold Packs; cool, wet towels; wearable personal cooling system) or passive cooling (e.g., physical rest, move to a cool environment, air-conditioned room, or shaded area) methods. This reduces body core temperature and allows for a more rapid “rehabilitation” during the rest break.”

Climate change and heat waves will have a major impact on individuals wearing Respirators, Surgical Masks, Elevation/Resistance Training Mask and/or Face Mask/Coverings. This is because Climate change will be contributing to increased heat-related illness and death to individuals throughout the world. Therefore, there is a need for a multifunctional face mask assembly embodiment comprising, the universal cool release vent harness sleeve/strap accessory that houses or secure a removable universal cool filter ice pack insert accessory to fastened to the face, head and neck, to provide a cool ambient microclimate around the face and/or aid in cooling the regulation of cerebral blood flow by means of the Hypothalamus which is responsible for regulating body temperature. Accordingly, “Climate Change Already Causes 5 Million Extra Deaths Per Year.” “Deaths caused by hot temperatures increased in all regions, which suggests that global warming due to climate change will lead to increases in heat-related deaths in the future, according to the new study.” (Robert Preidt, HealthDay Reporter, Climate Change Already Causes 5 Million Extra Deaths Per Year). Another article “How Heat Stress Has Become One of the Most Dangerous Hazards in the Workplace, Apr. 17, 2018” explains, “According to the CDC, over 600 people die from extreme heat every year. That number includes people of all ages, and occurs both at home and at work. It also indicates that in the same year, 16,320 workers suffered from heat-related illnesses that caused them to miss work. These deaths and heat-related illness are preventable. Take the necessary steps to avoid these incidents at your workplace.” Heat-related illnesses can affect all anyone throughout the world. These may include mail delivery, medical, chemical, industrial, construction, mining, landscaping, agricultural, military, sports, fitness, exercise, educational, recreational, leisure and home settings.

Furthermore, Congress passed a bill, “H.R.2193—Asuncion Valdivia Heat Illness and Fatality Prevention Act of 2021117th Congress (2021-2022)” “To direct the Occupational Safety and Health Administration to issue an occupational safety and health standard to protect workers from heat-related injuries and illnesses”. Some of their finding's list in the above mentioned bill includes but are not limited to; “Congress finds the following: (1) Excessive heat exposure poses a direct threat to workers and the economy. Climate change increases this danger, as 19 of the 20 hottest years on record have occurred since 2001. Rising temperatures are projected to cause an increase in heat-related workplace injuries and illnesses, a dramatic loss in labor capacity, and decreased productivity. (2) On average, 3 times as many people die from extreme heat in the United States each year than from hurricanes, floods, and tornadoes combined. (3) Heat-related illnesses can arise when high temperatures rise above the body's capacity to dispel heat. Impacts range from comparatively minor problems such as heat cramps to severe afflictions such as organ damage, heat exhaustion, stroke, and death. (4) Heat Stress And COVID-19 Are A Combined Threat To Workers. Respiratory infections, such as COVID-19, and protective equipment, like essential face masks, can increase susceptibility to heat exhaustion and heat stroke. Many of the symptoms of COVID-19 and heat stress may combine to cause acute illness that is especially deadly. (5) Jobs at highest risk of heat stress illness and death are disproportionately held by Black and Brown workers, a disparity that reflects the racial injustice inherent in our economic system. (6) Farmworkers and construction workers suffer the highest incidence of heat-related illness, but all outdoor and indoor workers employed in excessively hot and humid environments are at significant risk of material impairment of health or functional capacity. (11) Workers have a legal right to a safe workplace. The vast majority of heat-related workplace deaths and illnesses can be prevented by access to water, rest, and shade. Many employers don't provide these simple measures for workers according to the Occupational Safety and Health Administration.” Therefore, there is a need for a multifunctional face mask assembly embodiment comprising the universal cool release vent harness sleeve/strap accessory that houses or secure a removable universal cool filter ice pack insert accessory to fastened to the face, head and neck, to provide a cool ambient microclimate around the face and/or aid in cooling the regulation of cerebral blood flow by means of the Hypothalamus which is responsible for regulating body temperature.

Moreover, due to an ongoing rise in climate change and heat waves are likely to become more consistent and intense in the years to come. This will have a direct impact increasing the risk of heat-related illness. To help prevent heat-related illness among the general public, some states and local authorities opened cooling centers to temporarily address and potentially prevent heat-related illnesses during the presence of a heat wave. Some of the objectives of the Cooling centers is provide an air-conditioned cool environment, water, and restrooms. This service mainly targets individuals who are homeless, at-risk such as the elderly, and those without operable cooling systems at their place of residence. Currently, although these Cooling centers provide an air-conditioned environment, individuals still have to wear a mask/covering during their time there. If a person began to display any signs of a heat-related illness, traditional, conventional or modern respirators, surgical masks, elevation/resistance training mask and/or face mask/coverings should not be used to cool the individual temperature down. Therefore, a multifunctional face mask assembly embodiment comprising, the universal cool release vent harness sleeve/strap accessory that houses or secure a removable universal cool filter ice pack insert accessory to fastened to the face, head and neck, to provide a cool ambient microclimate around the face and/or aid in cooling the regulation of cerebral blood flow by means of the Hypothalamus which is responsible for regulating body temperature is needed.

Moreover, in 2011, OSHA launched a Heat Illness Prevention Program to educate employers and workers on the dangers of heat-related illnesses in the workplace. It was noted that millions of U.S. workers have heat exposures in the workplaces. Although it is clear that heat-related illnesses is preventable, every year, thousands suffer from occupational heat exposure, and some settings are deadly. “Under OSHA law, employers are responsible for providing workplaces free of known safety hazards. This includes protecting workers from extreme heat. An employer with workers exposed to high temperatures should establish a complete heat illness prevention program. Currently, OSHA recommends to “Allow workers to wear personal passive cooling items (e.g., icepack vests, cooling bandanas)”. Currently there is a need to provide workers with a multifunctional face mask assembly embodiment comprising, the universal cool release vent harness sleeve/strap accessory that houses or secure a removable universal cool filter ice pack insert accessory to fastened to the face, head and neck, to provide a cool ambient microclimate around the face and/or aid in cooling the regulation of cerebral blood flow by means of the Hypothalamus which is responsible for regulating body temperature.

Furthermore, because of climate change heat-related illnesses will continue among active-duty military soldiers, recruits, and reservists who routinely suffer from heat-related illness during deployments and/or field training. According to statistics maintained by the Military Health System, close to 3,000 military members experienced heat exhaustion or was hospitalized from having a heat stroke in 2018. Although there is cooling alternatives such as neck-cooling apparatus, cooling bandanas, cooling vests, and fabric comprising cooling micro-gels, that have been effectively used to reduce core temperature. However, they do not have a multifunctional face mask assembly embodiment comprising, the universal cool release vent harness sleeve/strap accessory that houses or secure a removable universal cool filter ice pack insert accessory to fastened to the face, head and neck region, to provide a cool ambient microclimate around the face and/or aid in cooling the regulation of cerebral blood flow by means of the Hypothalamus which is responsible for regulating body temperature.

Moreover, there is a need for a multifunctional face mask assembly embodiment comprising, the universal cool release vent harness sleeve/strap accessory that houses or secure a removable universal cool filter ice pack insert accessory to fastened to the face, head and neck region, to aid in intermittent cooling the regulation of cerebral blood flow by means of the Hypothalamus which is responsible for regulating body temperature. Intermittent per-cooling used during sports requiring high exertion, longer durations, and/or infrequent hydration breaks such as soccer, long distance running or lacrosse can help attenuate heat-related illness. There is an increase in exertional heat-related illness and injuries among those performing exertional activities or performances. This may include, but not limited to the following (e.g. football, outdoor golf, aerobics, basketball, cycling, running, crossfit, tennis, baseball etc.) According to the CDC, heat-related illness during practice or competition is the leading cause of death among U.S. high school athletes. Researchers also report that marching band musicians are at high risk of heat-related illness during afternoon long practices and playing on artificial turf put. Studies show that combined facial, head and neck cooling could reduce local skin temperature and improve local perceptual sensations including thermal sensation on athletes while exercising in the heat. Research also suggests the design of a portable facial, head and neck cooling systems is urgently required due to the great need to dissipate metabolic heat production during intensive sports activities. There is also a need for a multifunctional face mask assembly embodiment comprising, the universal cool release vent harness sleeve/strap accessory that houses or secure a removable universal cool filter ice pack insert accessory optionally fastened to the face, head and neck region to provide pre-cooling before exertional sport, recreational or activities, intermittent per-cooling used during to help improve performance, and post-cooling to aid in cooling the regulation of cerebral blood flow by means of the Hypothalamus which is responsible for regulating body temperature.

Furthermore, there is a need to provide a multifunctional face mask assembly embodiment comprising, the universal cool release vent harness sleeve/strap accessory that houses or secure a removable universal cool filter ice pack insert accessory to fastened to the face, head and neck, to provide a cool ambient microclimate around the face and aid in relieving a headache or migraine. A study revealed that the application of a frozen neck wrap at the onset of migraine pain was helpful in reducing pain for 77% of the study participants. Another study revealed that using a frozen neck wrap at the beginning of a migraine significantly reduced pain in participants with migraine headaches. Researchers believed placing cooling pack around the neck, cooled the blood flowing to the carotid artery in the neck. This helped to reduce the inflammation in the brain, which helped reduced the pain felt by migraines. traditional, conventional or modern respirators, surgical masks, elevation/resistance training mask and/or face mask/coverings do not provide ice cooling to aid in reducing headaches or migraines.

Furthermore, traditional, conventional or modern respirators, surgical masks, elevation/resistance training mask and/or face mask/coverings do not provide ice cooling to aid in reducing fever and/or hot flashes. Ice cooling is used to help alleviate a fever by means of lower body temperatures. traditional, conventional or modern respirators, surgical masks, elevation/resistance training mask and/or face mask/coverings are not recommended to wear during the onset stages of a fever. Moreover, traditional, conventional or modern respirators, surgical masks, elevation/resistance training mask and/or face mask/coverings do not provide comfort and cooling for a woman experiencing the onset of hot flashes in work settings. Therefore, there is a need to provide a multifunctional face mask assembly embodiment comprising, the universal cool release vent harness sleeve/strap accessory that houses or secure a removable universal cool filter ice pack insert accessory to fastened to the face, head and neck, to provide a cool ambient microclimate around the face and aid in relieving a fever and/or hot flashes.

Moreover, traditional, conventional or modern respirators, surgical masks, elevation/resistance training mask and/or face mask/coverings do not provide a mask sleeve/strap accessory to optionally house or secure handheld/portable Nebulizer mask within its housing. Neither do they provide a constant cool ambient microclimate around the face to aid in maintaining lower humidity temperatures within the face mask on hot and humid days. There are three main types of nebulizers; Jet, Ultrasonic and Mesh Nebulizers used for COPD and Asthma treatments. Neither do traditional, conventional or modern respirators, surgical masks, elevation/resistance training mask and/or face mask/coverings provide a multifunctional face mask assembly embodiment comprising, the universal cool release vent harness sleeve/strap accessory that houses or secure a removable universal cool filter ice pack insert accessory fastened to the face, head and neck, to aid in cooling the regulation of cerebral blood flow by means of the Hypothalamus which is responsible for regulating body temperature.

Furthermore, to help prevent heat-related illness and reduce high temperatures, traditional, conventional or modern respirators, surgical masks, elevation/resistance training mask and/or face mask/coverings do not provide a multifunctional face mask assembly embodiment comprising, the universal cool release vent harness sleeve/strap accessory that houses or secure a removable universal cool filter ice pack insert accessory that is interchangeably used as a helmet face covering while wearing a helmet. Therefore, there is a need for a multifunctional face mask assembly embodiment comprising, the universal cool release vent harness sleeve/strap accessory that houses or secure a removable universal cool filter ice pack insert accessory that when placed on the face, head and neck provides aid in cooling the regulation of cerebral blood flow by means of the Hypothalamus which is responsible for regulating body temperature. Some of these helmets include the following (e.g., firefighting, Safety Helmet, Tactical, Football, Baseball, Lacrosse, Protective Headgear, Hockey, Rugby Headgear, Skateboard Helmet, Open Face Helmet, Motorcycle and/or Bike Helmet etc.). The multifunctional face mask assembly embodiment comprising, the universal cool release vent harness sleeve/strap accessory and the removable universal cool filter ice pack insert accessory can be interchangeably used for pre-cooling before work, intermittent per-cooling during breaks or post-cooling after work.

### SUMMARY OF THE INVENTION

In view of the above content, a multifunctional face mask assembly embodiment comprising a Filtrator™ nose cup accessory, a cartridge filter adapter, a filter cover with air vents, a filter, a universal cool release vent harness sleeve/strap accessory, a removable universal cool filter ice pack insert accessory, a universal cool filter ice pack insert pouch accessory. A multifunctional face mask assembly embodiment fastened to aid in lowering skin temperature around the face, head and neck, to provide cool ambient microclimate around the face, and/or aid in cooling the regulation of cerebral blood flow by means of the Hypothalamus which is responsible for regulating body temperature.

The present invention in summary, pertains to a multifunctional face mask assembly embodiment comprising; a Filtrator™ nose cup accessory. The Filtrator™ nose cup accessory comprises a plurality of cartridge filter adapters, a filter cover with air vents and a filter. The cartridge filter adapters are used to hold or secure cartridge filters to its embodiment. The filter cover with air vents is used to allow filtered ventilation to be released from the housing or microclimate by means of a filter. The filter allows filtration from ones breathing to be released through the Filtrator™ nose cup accessory. The Filtrator™ nose cup accessory is housed, secured, or attached to the multifunctional face mask assembly embodiment by means of the universal cool release vent respirator harness sleeve/strap accessory. The Filtrator™ nose cup accessory comprises a cavity or opening used to interchangeably house, secure a removable universal cool filter ice pack insert accessory within its housing. The Filtrator™ nose cup accessory allows the removable universal cool filter ice pack insert accessory to provide a cool ambient microclimate around the face. Therefore, providing aid in reducing humidity, moisture, heat and carbon dioxide levels.

The present invention is unique compared to traditional, conventional or modern respirators, surgical masks, elevation/resistance training mask and face mask/coverings in that the invention comprises a multifunctional face mask assembly embodiment comprising; a universal cool release vent harness sleeve/strap accessory. The universal cool release vent harness sleeve/strap accessory comprises a cavity opening use to house or secure various nose cup or breathing treatment mask embodiments (e.g., respirators, nebulizers, silicone-based mask, elevation/resistance training mask). The universal cool release vent harness sleeve/strap accessory is optionally used to house or secure a elastomeric half facepiece reusable respirator, resistance training mask, dust particle mask or face mask. The universal cool release vent harness sleeve/strap accessory is optionally used to aid in improving performance by means of pre-cooling, intermittent cooling, per/mid-cooling or post-cooling during exertional sports, fitness and/or exercises. The universal cool release vent harness sleeve/strap accessory comprises a plurality of pouches attached on its inner surface that comprises a plurality of cool release vents to allow a faster cool released sensation from the removable universal cool filter ice pack insert accessory housed or secured within its pouch. The universal cool release vent harness sleeve/strap accessory allows the removable universal cool filter ice pack insert accessory to fastened to the face, head and neck, to provide a cool ambient microclimate around the face and/or aid in cooling the regulation of cerebral blood flow by means of the Hypothalamus which is responsible for regulating body temperature. The universal cool release vent harness sleeve/strap accessory comprises a plurality of straps comprising upper straps, middle straps and lower straps used to attach, fasten or secure the universal cool release vent harness sleeve/strap accessory to the head. The upper straps are fashioned to be wrapped, placed, contoured, secured or fixed around the top of the head with the use of separable fasteners. The middle straps are fashioned to be wrapped, placed, contoured, secured or fixed around the back of the head behind the ears. The middle straps are securely coupled or joined to both upper straps and lower straps. The lower straps are fashioned to be wrapped, placed, contoured, secured or fixed around the back of the neck for placement on the nape of the neck or Feng Fu pressure point with the use of separable fasteners.

The present invention is unique compared to traditional, conventional or modern Respirators, Surgical Masks, Elevation/Resistance Training Mask and Face Mask/Coverings in that the multifunctional face mask assembly embodiment comprises a removable universal cool filter ice pack insert accessory to maintain a cool microclimate within the multifunctional face mask assembly embodiment Filtrator™ nose cup accessory. The removable universal cool filter ice pack insert accessory provides a constant cooling effect within the Filtrator™ nose cup accessory by means of cool ambient microclimate around the face. Therefore, providing aid in reducing humidity, moisture, heat and carbon dioxide levels. Thus, reducing the ambient thermal temperature surrounding the wearer's nose and mouth. The removable universal cool filter ice pack insert accessory also aids in providing fresh cool air to breathe within the microclimate. Also, unlike traditional, conventional or modern Respirators, Surgical Masks, Elevation/Resistance Training Mask and Face Mask/Coverings, the present invention can aid in providing temporary aid to individuals with chronic respiratory conditions such as COPD or Asthma breathe more comfortable within a cool microclimate multifunctional face mask assembly embodiment on hot and humid days. The removable universal cool filter ice pack insert accessory can be constructed or configured into a plurality of shapes or forms for placement within traditional, conventional or modern Respirators, Surgical Masks, Elevation/Resistance Training Mask and Face Mask/Coverings.

Furthermore. the present invention is unique compared to traditional, conventional or modern Respirators, Surgical Masks, Elevation/Resistance Training Mask and Face Mask/Coverings in that the multifunctional face mask assembly embodiment comprises a universal cool filter ice pack insert pouch accessory to provide cool ambient microclimate around the face, to aid in lowering skin temperature around the face and/or head by means of the removable universal cool filter ice pack insert accessory. The universal cool filter ice pack insert pouch accessory comprises a plurality of cool release vents cavity opening on its top surface to allow a faster cool released sensation from the removable universal cool filter ice pack insert accessory housed or secured within its pouch. The universal cool filter ice pack insert pouch accessory allows the removable universal cool filter ice pack insert accessory to provide a cool ambient microclimate around the face and/or head to aid in cooling the regulation of cerebral blood flow by means of the Hypothalamus which is responsible for regulating body temperature. The universal cool filter ice pack insert pouch accessory comprises a pocket flap on its bottom surface used to house, secure, store or hold a removable universal cool filter ice pack insert accessory within the universal cool filter ice pack insert pouch accessory housing. The universal cool filter ice pack insert pouch accessory comprises an absorbent moisture-wicking and breathable material used to aid in absorbing droplets and sweat. The universal cool filter ice pack insert pouch accessory can be constructed or configured into a plurality of shapes or forms for placement within the multifunctional face mask assembly embodiment Filtrator™ nose cup accessory and/or traditional, conventional or modern Respirators, Surgical Masks, Elevation/Resistance Training Mask and Face Mask/Coverings.

Furthermore. the present invention is unique compared to traditional, conventional or modern Respirators, Surgical Masks, Elevation/Resistance Training Mask and Face Mask/Coverings in that the present invention provides an apparatus comprising; a multifunctional face mask assembly embodiment fastened to aid in lowering skin temperature around the face, head and neck, to provide cool ambient microclimate around the face, and/or aid in cooling the regulation of cerebral blood flow by means of the Hypothalamus which is responsible for regulating body temperature. The present invention helps prevent or reduce heat-related illnesses in the following settings; those who work or reside in hot environments (e.g., indoor/outdoor emergency settings, industrial workers, agriculture, farmworkers, landscaping, spray painters, welders, miners, construction workers, first responders, fireman, law enforcement officers and/or riot control); women who suffer from hot flashes; individuals who suffer from headaches and/or migraines; Cooling Centers helping individuals seeking relief to help prevent potential heat-related illness; Military Recruits, Reservist or Field Training exercise or activities; individuals using handheld/portable Nebulizer mask (e.g., COPD and/or Asthma users); individuals performing exertional sports, fitness, recreational, exercise (e.g. football, resistance fitness training, outdoor golf, aerobics, basketball, cycling, running, crossfit, tennis, baseball etc.). The present invention is used as a Cyro-Cooling mask to aid in relieving stress in home, office, medical, recreation and/or leisure settings.

### BRIEF DESCRIPTION OF THE DRAWINGS

FIG. 1A is a top view of the present invention displaying a removable universal cool filter ice pack insert accessory displaying heat-sealed lines.

FIG. 1B is a top view of the present invention displaying a universal cool filter ice pack insert pouch accessory comprising a plurality of cool release vents.

FIG. 1C is a rear view of a resistance training mask cavity housing or storing a removable universal cool filter ice pack insert accessory.

FIG. 1D is a top view of the present invention displaying a removable universal cool filter ice pack insert accessory displaying heat-sealed lines. FIG. 1B is a rear view of a face mask displaying a universal cool filter ice pack insert pouch accessory with cool vents.

FIG. 1E is a top view of the present invention displaying a universal cool filter ice pack insert pouch accessory comprising a plurality of cool release vents.

FIG. 1F is a rear view of a silicone mask with filter cavity housing or storing a removable universal cool filter ice pack insert accessory.

FIG. 2A is a front view showing the front outer cover of the present invention universal cool release vent harness sleeve/strap accessory comprising an open cavity with separable fasteners around its outer surface to attach or secure a filter cover pouch.

FIG. 2B is a front view showing the front outer cover of the present invention universal cool release vent harness sleeve/strap accessory comprising a filter cover pouch being affixed, secured or attached to an open cavity by means of separable fasteners around its outer surface.

FIG. 2C is a top view of the present invention displaying a removable universal cool filter ice pack insert accessory and a reverse side of the filter cover pouch comprising separable fasteners around its outer surface.

FIG. 2D is a side view showing the present invention universal cool release vent harness sleeve/strap accessory comprising separable fasteners around the mouth cavity, head and neck straps.

FIG. 3A is a rear view sample of a 3M Elastomeric Half Facepiece Reusable Respirator with cartridges, housing or storing a removable universal cool filter ice pack insert accessory.

FIG. 3B is a rear view sample of a silicone mask with filter, housing or storing a removable universal cool filter ice pack insert accessory.

FIG. 3C is a rear view sample of a MSA Elastomeric Half Facepiece Reusable Respirator with cartridges, housing or storing a removable universal cool filter ice pack insert accessory.

FIG. 3D is a rear view sample of a 3M full facepiece reusable respirator with cartridges, housing or storing a removable universal cool filter ice pack insert accessory.

FIG. 3E is a rear view sample of a Avon M50/FM50 gas mask respirator with cartridges, housing or storing a removable universal cool filter ice pack insert accessory.

FIG. 4A is a front perspective view of the present invention displaying the Filtrator™ nose cup accessory, cartridge filter adapter and filter cover with air vents.

FIG. 4B is a front perspective view of the present invention comprising the universal cool release vent respirator harness sleeve/strap accessory, universal cool release vent respirator harness sleeve/strap accessory, filter cover with air vents and plurality cartridge filter adapters securing two cartridge filters.

FIG. 4C is a front perspective view of the present invention displaying the Filtrator™ nose cup accessory, cartridge filter adapter and filter bracket holder with air vents.

FIG. 4D is a top perspective view of the present invention displaying the reverse or opposite side of the Filtrator™ nose cup accessory filter cover with air vents.

FIG. 4E is a side perspective view of the present invention displaying the Filtrator™ nose cup accessory housing an outlined removable universal cool filter ice pack insert accessory, a filter insert and cartridge filter adapter.

FIG. 5A is a top perspective view of the present invention displaying a removable universal cool filter ice pack insert accessory displaying heat-sealed lines.

FIG. 5B is a top perspective view of the present invention displaying a removable universal cool filter ice pack insert accessory displaying heat-sealed lines.

FIG. 5C is a top perspective view of the present invention displaying a removable universal cool filter ice pack insert accessory displaying heat-sealed lines.

FIG. 5D is a top perspective view of the present invention displaying a removable universal cool filter ice pack insert accessory displaying heat-sealed lines.

FIG. 5E is a top perspective view of the present invention displaying a removable universal cool filter ice pack insert accessory displaying heat-sealed lines.

FIG. 5F is a top perspective view of the present invention displaying a removable universal cool filter ice pack insert accessory displaying heat-sealed lines.

FIG. 6A is a top view of the present invention displaying a universal cool filter ice pack insert pouch accessory comprising a plurality of cool release vents and separable fasteners.

FIG. 6B is a top view of the present invention displaying a universal cool filter ice pack insert pouch accessory comprising a plurality of cool release vents and separable fasteners.

FIG. 6C is a top view of the present invention displaying a universal cool filter ice pack insert pouch accessory comprising a plurality of cool release vents and separable fasteners.

FIG. 6D is a top view of the present invention displaying a universal cool filter ice pack insert pouch accessory comprising a plurality of cool release vents and separable fasteners.

FIG. 6E is a top view of the present invention displaying a universal cool filter ice pack insert pouch accessory comprising a plurality of cool release vents and separable fasteners.

FIG. 7A is a front view of the present invention displaying a universal cool filter ice pack insert pouch accessory securely attached to a MSA Ultra Elite® Full-Facepiece Respirator Head Harness by means of separable fasteners.

FIG. 7B is a front view of the present invention displaying a universal cool filter ice pack insert pouch accessory securely attached to an Avon M50/FM50 CBRN Gas Mask Head Harness by means of separable fasteners.

FIG. 7C is a front view of the present invention displaying a universal cool filter ice pack insert pouch accessory securely attached to a Scott AV-3000 SCBA Head Harness by means of separable fasteners.

FIG. 7D is a front view of the present invention displaying a universal cool filter ice pack insert pouch accessory securely attached to a 3M™ Head Harness FF-400-04 by means of separable fasteners.

FIG. 7E is a rear perspective view of the present invention displaying a universal cool filter ice pack insert pouch accessory securely attached to a 3M™ Head Harness FF-400-04 by means of separable fasteners.

FIG. 8A is a rear perspective view of the present invention displaying a universal cool filter ice pack insert pouch accessory securely attached to an Elastomeric Half Facepiece Reusable Respirator Head Harness by means of separable fasteners.

FIG. 8B is a front perspective view of the present invention displaying a universal cool filter ice pack insert pouch accessory securely attached to an Elastomeric Half Facepiece Reusable Respirator Head Harness by means of separable fasteners.

FIG. 8C is a top perspective view of the present invention displaying a removable universal cool filter ice pack insert accessory displaying heat-sealed lines.

FIG. 8D is a rear perspective view of the present invention displaying a universal cool filter ice pack insert pouch accessory securely attached to an Elastomeric Full Facepiece Reusable Respirator Head Harness by means of separable fasteners.

FIG. 8E is a top view of the present invention displaying a universal cool filter ice pack insert pouch accessory comprising a plurality of cool release vents and separable fasteners.

FIG. 8F is a top perspective view of the present invention displaying a removable universal cool filter ice pack insert accessory displaying heat-sealed lines.

FIG. 9A is a top view of the present invention displaying a universal cool filter ice pack insert pouch accessory comprising a plurality of cool release vents and separable fasteners.

FIG. 9B is a top view of the present invention displaying a universal cool filter ice pack insert pouch accessory securely attached to a 3M™ Comfort Cradle Head Harness Attachment FF-400-06 by means of separable fasteners.

FIG. 9C is a rear view of the present invention displaying a universal cool filter ice pack insert pouch accessory securely attached to a 3M™ Comfort Cradle Head Harness Attachment FF-400-06 by means of separable fasteners.

FIG. 9D is a top view of the present invention displaying a universal cool filter ice pack insert pouch accessory comprising a plurality of cool release vents and separable fasteners.

FIG. 9E is a front view of the present invention displaying a universal cool filter ice pack insert pouch accessory securely attached to a 3M Full Facepiece Respirator 6000 Series Head Harness by means of separable fasteners.

FIG. 9F is a rear view of the present invention displaying a universal cool filter ice pack insert pouch accessory securely attached to a 3M Full Facepiece Respirator 6000 Series Head Harness by means of separable fasteners.

FIG. 10A is a side view showing the reverse side of the present invention universal cool release vent harness sleeve/strap accessory pouch inserts comprising a plurality of cool release vents to aid in cooling the face, head and neck.

FIG. 10B is a side view showing the reverse side of the present invention universal cool release vent respirator harness sleeve/strap accessory comprising a plurality of cool release vents and cartridge filter adapter cavity.

FIG. 10C is a side view showing the reverse side of the present invention universal cool release vent harness sleeve/strap accessory pouch inserts comprising a plurality of cool release vents to aid in cooling the face, head and neck and displaying the removable universal cool filter ice pack insert accessory on its surface.

FIG. 10D is a side view showing the reverse side of the present invention universal cool release vent respirator harness sleeve/strap accessory displaying the removable universal cool filter ice pack insert accessory on its surface and cartridge filter adapter cavity.

FIG. 11A is a top view of the present invention universal cool release vent harness sleeve/strap comprising a plurality of cool release vents and a cavity opening.

FIG. 11B is a reverse top view of the present invention universal cool release vent harness sleeve/strap comprising a plurality of cool release vents while housing a mask with filter.

FIG. 11C is a top view of the present invention universal cool release vent respirator harness sleeve/strap accessory comprising a plurality of cool release vents and a cavity opening.

FIG. 11D is a front view of the present invention multifunctional face mask assembly embodiment comprising the universal cool release vent respirator harness sleeve/strap accessory, Filtrator™ nose cup accessory, filter cover and cartridge filter adapter, filter insert not shown.

FIG. 11E is a side view showing the reverse side of the present invention universal cool release vent respirator harness sleeve/strap accessory comprising a plurality of cool release vents, housing a elastomeric half facepiece reusable respirator.

FIG. 12A is a side view showing the reverse side of the present invention universal cool release vent harness sleeve/strap accessory comprising a plurality of cool release vents while housing a resistance training mask.

FIG. 12B is a side view showing the reverse side of the present invention universal cool release vent respirator harness sleeve/strap accessory comprising a plurality of cool release vents while housing a Nebulizer mask.

FIG. 12C is a side view showing the reverse side of the present invention universal cool release vent respirator harness sleeve/strap accessory comprising a plurality of cool release vents while housing an elastomeric half facepiece reusable respirator with cartridges.

FIG. 12D is a side view displaying the present invention universal cool release vent harness sleeve/strap accessory cooling effects on the face, head, and neck carotid artery and internal jugular vein.

FIG. 12E is a back view displaying the present invention universal cool release vent harness sleeve/strap accessory cooling effects on head, neck, Circle of Willis arteries and feng fu point/nape of the neck.

FIG. 13A is a front view showing the reverse side of the present invention universal cool release vent harness sleeve/strap accessory being used for pre-cooling, intermittent per/mid-cooling or post cooling comprising a plurality of cool release vents around the face, head, and neck, filter cover pouch not shown.

FIG. 13B is a side view showing the reverse side of the present invention universal cool release vent harness sleeve/strap accessory pouch inserts comprising a plurality of cool release vents to aid in cooling the face, head and neck.

FIG. 13C is a front view showing the reverse side of the present invention universal cool release vent harness sleeve/strap accessory being used as a HELMET FACE COVERING for pre-cooling, intermittent per/mid-cooling or post cooling and comprising a plurality of cool release vents around the face. Filter cover pouch not shown.

FIG. 13D is a side view showing the reverse side of the present invention universal cool release vent harness sleeve/strap accessory being used as a HELMET FACE COVERING for pre-cooling, intermittent per/mid-cooling or post cooling and comprising a plurality of cool release vents around the face, head and neck. Filter cover pouch not shown.

### DETAILED DESCRIPTION OF THE INVENTION

The present invention provides an apparatus comprising; a multifunctional face mask assembly embodiment comprising a Filtrator™ nose cup accessory, a cartridge filter adapter, a filter cover with air vents, a filter, a universal cool release vent harness sleeve/strap accessory, a removable universal cool filter ice pack insert accessory, a universal cool filter ice pack insert pouch accessory. A multifunctional face mask assembly embodiment fastened to aid in lowering skin temperature around the face, head and neck, to provide cool ambient microclimate around the face, and/or aid in cooling the regulation of cerebral blood flow by means of the Hypothalamus which is responsible for regulating body temperature. The present invention helps reduce heat-related illnesses among some the following; those who work or reside in hot environments (e.g., indoors or outdoors), women who suffer from hot flashes, individuals who suffer from headaches and/or migraines, individuals who use handheld/portable Nebulizer mask (e.g., COPD and/or Asthma), individuals performing exertional sports, fitness, recreational, exercise, home, office, medical and/or leisure settings.

The present invention is unique compared to traditional, conventional or modern Respirators, Surgical Masks, Elevation/Resistance Training Mask and Face Mask/Coverings in that the present invention provides a multifunctional face mask assembly embodiment comprising; a Filtrator™ nose cup accessory. The Filtrator™ nose cup accessory comprises a plurality of cartridge filter adapters, a filter cover with air vents and a filter. The cartridge filter adapters are used to hold or secure cartridge filters to its embodiment. The filter cover with air vents is used to allow filtered ventilation to be released from the housing or microclimate by means of a filter. The filter allows filtration from ones breathing to be released through the Filtrator™ nose cup accessory. The Filtrator™ nose cup accessory is housed, secured, or attached to the multifunctional face mask assembly embodiment by means of the universal cool release vent harness sleeve/strap accessory. The Filtrator™ nose cup accessory comprises a cavity or opening used to interchangeably house, secure a removable universal cool filter ice pack insert accessory within its housing. The Filtrator™ nose cup accessory allows the removable universal cool filter ice pack insert accessory to provide a cool ambient microclimate around the face. Therefore, providing aid in reducing humidity, moisture, heat and carbon dioxide levels.
The present invention is unique compared to traditional, conventional or modern Respirators, Surgical Masks, Elevation/Resistance Training Mask and Face Mask/Coverings in that the present invention provides a multifunctional face mask assembly embodiment comprising; a universal cool release vent harness sleeve/strap accessory. The universal cool release vent harness sleeve/strap accessory comprises a cavity opening use to house or secure various nose cup mask or breathing treatment mask embodiments (e.g., nebulizers, silicone-based mask). The universal cool release vent harness sleeve/strap accessory is optionally used to house or secure a elastomeric half facepiece reusable respirator, resistance training mask, dust particle mask or face mask (e.g., 3M half facepiece respirators, MSA half facepiece respirators, Training Mask 2.0, 3.0, Apollo and VENT Filtration Mask). The universal cool release vent harness sleeve/strap accessory is optionally used to aid in improving performance by means of pre-cooling, intermittent cooling, per/mid-cooling or post-cooling during exertional sports, fitness and/or exercises. The universal cool release vent harness sleeve/strap accessory comprises a plurality of pouches attached on its inner surface that comprises a plurality of cool release vents cavity opening to allow a faster cool released sensation from the removable universal cool filter ice pack insert accessory housed or secured within its pouch. The universal cool release vent harness sleeve/strap accessory allows the removable universal cool filter ice pack insert accessory to fastened to the face, head and neck, to provide a cool ambient microclimate around the face and/or aid in cooling the regulation of cerebral blood flow by means of the Hypothalamus which is responsible for regulating body temperature. The universal cool release vent harness sleeve/strap accessory comprises a plurality of straps comprising upper straps, middle straps and lower straps used to attach, fasten or secure the universal cool release vent harness sleeve/strap accessory to the head. The upper straps are fashioned to be wrapped, placed, contoured, secured or fixed around the top of the head with the use of separable fasteners. The middle straps are fashioned to be wrapped, placed, contoured, secured or fixed around the back of the head behind the ears. The middle straps are securely coupled or joined to both upper straps and lower straps. The lower straps are fashioned to be wrapped, placed, contoured, secured or fixed around the back of the neck for placement on the nape of the neck or Feng Fu pressure point with the use of separable fasteners.
The present invention is unique compared to traditional, conventional or modern Respirators, Surgical Masks, Elevation/Resistance Training Mask and Face Mask/Coverings in that the present invention provides a multifunctional face mask assembly embodiment comprising; a removable universal cool filter ice pack insert accessory to maintain a cool microclimate within the multifunctional face mask assembly embodiment Filtrator™ nose cup accessory. The removable universal cool filter ice pack insert accessory provides a constant cooling effect within the Filtrator™ nose cup accessory by means of cool ambient microclimate around the face. Therefore, providing aid in reducing humidity, moisture, heat and carbon dioxide levels. Thus, reducing the ambient thermal temperature surrounding the wearer's nose and mouth. The removable universal cool filter ice pack insert accessory also aids in providing fresh cool air to breathe within the microclimate. Also, unlike traditional, conventional or modern Respirators, Surgical Masks, Elevation/Resistance Training Mask and Face Mask/Coverings, the present invention can aid in providing temporary aid to individuals with chronic respiratory conditions such as COPD or Asthma breathe more comfortable within a cool microclimate multifunctional face mask assembly embodiment on hot and humid days. The removable universal cool filter ice pack insert accessory can be constructed or configured into a plurality of shapes or forms for placement within traditional, conventional or modern Respirators, Surgical Masks, Elevation/Resistance Training Mask and Face Mask/Coverings (e.g., 3M Company, Avon Protection Systems, Inc., Honeywell International, Inc., MSA Safety Inc., Drägerwerk AG & Co. KG, KOWA, Uvex, Halyard Healthcare, McKesson, Molnlycke Health, Hakugen, Shanghai Dasheng, Sinotextiles, Irema and/or Winner Medical).
The present invention is unique compared to traditional, conventional or modern Respirators, Surgical Masks, Elevation/Resistance Training Mask and Face Mask/Coverings in that the present invention provides a multifunctional face mask assembly embodiment comprising; a universal cool filter ice pack insert pouch accessory to provide cool ambient microclimate around the face, to aid in lowering skin temperature around the face and/or head by means of the removable universal cool filter ice pack insert accessory. The universal cool filter ice pack insert pouch accessory comprises a plurality of cool release vents cavity opening on its top surface to allow a faster cool released sensation from the removable universal cool filter ice pack insert accessory housed or secured within its pouch. The universal cool filter ice pack insert pouch accessory allows the removable universal cool filter ice pack insert accessory to provide a cool ambient microclimate around the face and/or head to aid in cooling the regulation of cerebral blood flow by means of the Hypothalamus which is responsible for regulating body temperature. The universal cool filter ice pack insert pouch accessory comprises a pocket flap on its bottom surface used to house, secure, store or hold a removable universal cool filter ice pack insert accessory within the universal cool filter ice pack insert pouch accessory housing. The universal cool filter ice pack insert pouch accessory comprises an absorbent moisture-wicking and breathable material used to aid in absorbing droplets and sweat. The universal cool filter ice pack insert pouch accessory can be constructed or configured into a plurality of shapes or forms for placement within the multifunctional face mask assembly embodiment Filtrator™ nose cup accessory and/or traditional, conventional or modern Respirators, Surgical Masks, Elevation/Resistance Training Mask and Face Mask/Coverings (e.g., 3M Company, Avon Protection Systems, Inc., Honeywell International, Inc., MSA Safety Inc., Drägerwerk AG & Co. KG, KOWA, Uvex, Halyard Healthcare, McKesson, Molnlycke Health, Hakugen, Shanghai Dasheng, Sinotextiles, Irema and/or Winner Medical).
Furthermore, the present invention is unique compared to traditional, conventional or modern Respirators, Surgical Masks, Elevation/Resistance Training Mask and Face Mask/Coverings in that the present invention provides a multifunctional face mask assembly embodiment to aid in preventing heat-related illness and reduce high temperatures, by means of a the universal cool release vent harness sleeve/strap accessory that houses or secure a removable universal cool filter ice pack insert accessory used interchangeably as a helmet face covering while wearing a helmet. The universal cool release vent harness sleeve/strap accessory that houses or secure a removable universal cool filter ice pack insert accessory that when placed on the face, head and neck provides aid in cooling the regulation of cerebral blood flow by means of the Hypothalamus which is responsible for regulating body temperature. Some of these helmets include the following (e.g., firefighting, Safety Helmet, Tactical, Football, Baseball, Lacrosse, Protective Headgear, Hockey, Rugby Headgear, Skateboard Helmet, Open Face Helmet, Motorcycle and/or Bike Helmet etc.). The multifunctional face mask assembly embodiment comprising, the universal cool release vent harness sleeve/strap accessory and the removable universal cool filter ice pack insert accessory can be interchangeably used for pre-cooling before work, intermittent per-cooling during breaks or post-cooling after work.
The present invention provides an apparatus comprising; a multifunctional face mask assembly embodiment comprising a Filtrator™ nose cup accessory, a cartridge filter adapter, a filter cover with air vents, a filter, a universal cool release vent harness sleeve/strap accessory, a removable universal cool filter ice pack insert accessory, a universal cool filter ice pack insert pouch accessory. A multifunctional face mask assembly embodiment fastened by means of separable fasteners to provide aid in lowering skin temperature around the face, head and neck, to provide cool ambient microclimate around the face, and/or aid in cooling the regulation of cerebral blood flow by means of the Hypothalamus which is responsible for regulating body temperature. The present invention helps prevent or reduce heat-related illnesses in the following settings; those who work or reside in hot environments (e.g., indoor/outdoor emergency settings, industrial workers, agriculture, farmworkers, landscaping, spray painters, welders, miners, construction workers, first responders, fireman, law enforcement officers and/or riot control); women who suffer from hot flashes; individuals experiencing the onset of a fever; individuals who suffer from headaches and/or migraines; used in Cooling Centers to lower skin temperatures around the face, head and neck helping individuals seeking relief from potential heat-related illnesses; Military Recruits, Reservist or Field Training exercise or activities; individuals using handheld/portable Nebulizer mask (e.g., COPD and/or Asthma users); individuals performing exertional sports, fitness, recreational, exercise (e.g. football, resistance fitness training, outdoor golf, aerobics, basketball, cycling, running, crossfit, tennis, baseball etc.). The present invention is used as a Cyro-Cooling mask to aid in relieving stress in home, office, medical, recreation and/or leisure settings.

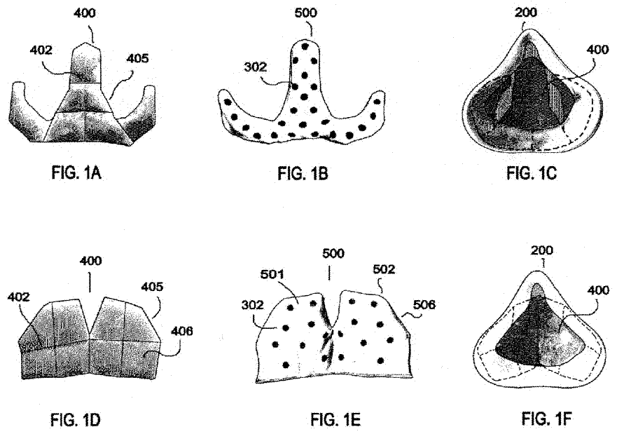

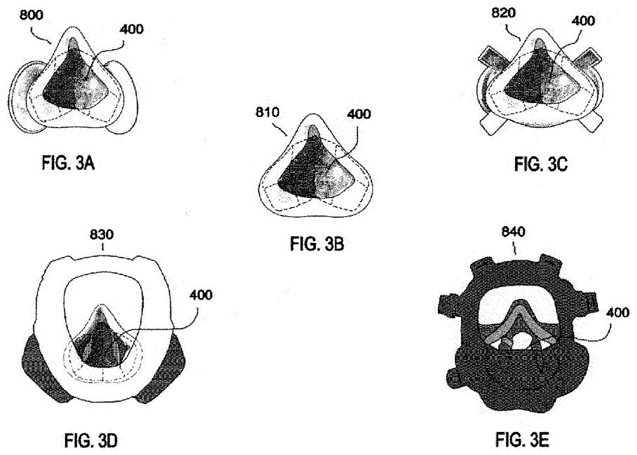

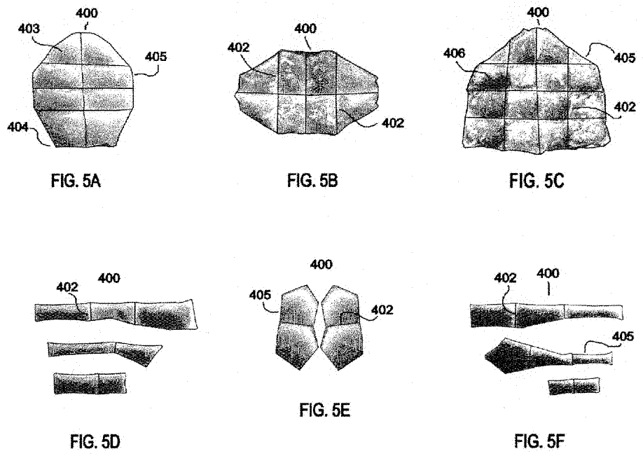

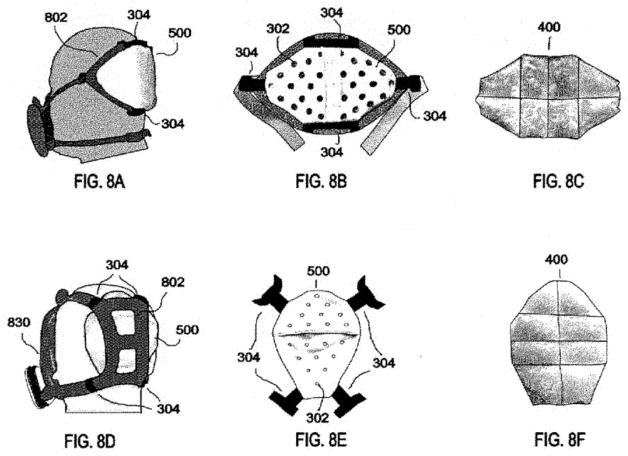

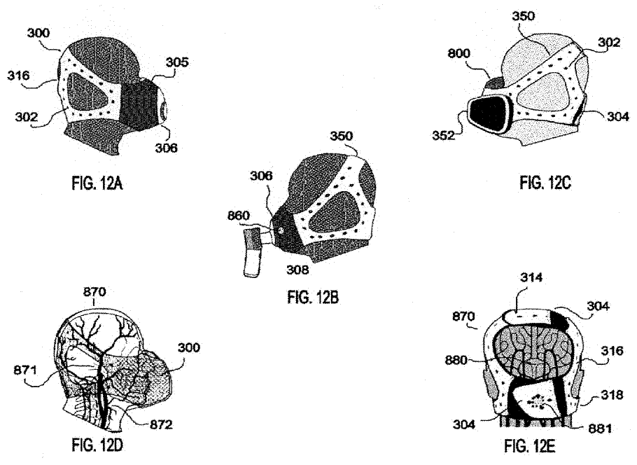

Referring now to the drawings FIGS. 1A-1F, 2C, 3A-3E, 4E, 5A-5F, 8A-8B, 8D-8E, 10C, 12A, 12D and 12E. The multifunctional face mask assembly is generally denoted by the reference numeral 100. The multifunctional face mask assembly 100 comprising a removable universal cool filter ice pack insert accessory 400. The removable universal cool filter ice pack insert accessory 400 is conformed, constructed or configured into a plurality of shapes as seen in FIGS. 1A, 1D, 2C and 5A-5F. The removable universal cool filter ice pack insert accessory 400 generally have a top surface 403 and a bottom surface 404 as seen in FIGS. 1A, 1D, 2C and 5A-5F. The top surface 403 and the bottom surface 404 are coupled or joined by their perimeter edges 405 as best seen in FIGS. 5A-5F to form a sealed pouch 406 filled with a non-toxic water-soluble gel filling material, substance or compounds as seen in FIGS. 1A, 1D, 2C and 5A-5F. The removable universal cool filter ice pack insert accessory 400 comprises heat-sealed lines 402 configured to allow pliable bending to be optionally nested within the sleeve strap pouch insert 312, nose cup housing of a respirator, elevation/resistance training mask, and face mask/covering to provide a cool ambient microclimate around the face as best seen in FIGS. 3A-3E. FIG. 3A shows the removable universal cool filter ice pack insert accessory 400 interchangeably nested or stored in the nose cup of a 3M Elastomeric Half Facepiece Reusable Respirator with cartridges 800. FIG. 3B shows the removable universal cool filter ice pack insert accessory 400 interchangeably nested or stored in the nose cup of a silicone mask with filter 810. FIG. 3C shows the removable universal cool filter ice pack insert accessory 400 interchangeably nested or stored in the nose cup of a MSA Elastomeric Half Facepiece Reusable Respirator with cartridges 820. FIG. 3D shows the removable universal cool filter ice pack insert accessory 400 interchangeably nested or stored in the nose cup of a 3M full facepiece reusable respirator with cartridges 830. FIG. 3E shows the removable universal cool filter ice pack insert accessory 400 interchangeably nested or stored in the nose cup of a Avon M50/FM50 gas mask respirator with cartridges 840. As seen in FIGS. 5A-5C shows a plurally of removable universal cool filter ice pack insert accessories 400 optionally used to aid in cooling the back of the head by means of the universal cool filter ice pack insert pouch accessory 500. Seen in FIGS. 8B and 8E shows the removable universal cool filter ice pack insert accessory 400 housed and secured within the housing of the universal cool filter ice pack insert pouch accessory 500 by means of separable fasteners 304, revealing the universal cool filter ice pack insert pouch accessory 500 cool release vents 302 cavity opening. FIG. 8A shows the housing of the universal cool filter ice pack insert pouch accessory 500 interchangeably attached, fastened or secured by means of separable fasteners 304 being contoured around the back of the head to the head harness 802 of a 3M Elastomeric Half Facepiece Reusable Respirator with cartridges 800. FIG. 8D shows the housing of the universal cool filter ice pack insert pouch accessory 500 interchangeably attached, fastened or secured by means of separable fasteners 304 being contoured around the back of the head to the head harness 802 of a full facepiece respirator 830. As seen in FIGS. 5D and 5F, shows a plurally of removable universal cool filter ice pack insert accessory 400 conformed or constructed to be optionally contoured around the head and neck by means of the universal cool release vent harness sleeve/strap accessory 300 as best seen in FIGS. 12A and 12E. As seen in FIG. 12D, shows a skeletal diagram 870 with the removable universal cool filter ice pack insert accessory 400 housed or secured in the universal cool release vent harness sleeve/strap accessory 300 covering, tracking or crossing the internal carotid artery 871 and external carotid arteries 872 providing aid in lowering skin temperature around the face, head and neck, to aid in cooling the regulation of cerebral blood flow by means of the Hypothalamus which is responsible for regulating body temperature. As seen in FIG. 12E, shows the back head of a skeletal diagram 870 with the removable universal cool filter ice pack insert accessory 400 housed or secured in the universal cool release vent harness sleeve/strap accessory 300 tracking forming a circle around the back of the head providing cooling to the Circle of Willis arteries 880 and cooling to the feng fu point/nape of the neck 881 providing aid in lowering skin temperature around the head and neck, to aid in cooling the regulation of cerebral blood flow by means of the Hypothalamus which is responsible for regulating body temperature.

The removable universal cool filter ice pack insert accessory 400 can be shaped, constructed or configured into a plurality of shapes or forms comprising such as without limitation; a rectangular, square, circle, heart, clover, star, diamond, oval, octagon, trapezoid, pentagon, hexagon, triangle, parallelogram, rhombus or unique shape that is conformed, adaptable, fashioned, designed or shaped for placement within traditional, conventional or modern Respirators, Surgical Masks, Elevation/Resistance Training Mask and Face Mask/Coverings. The removable universal cool filter ice pack insert accessory 400 can be shaped, constructed or configured into unique shapes that conforms traditional, conventional or modern Respirators, Surgical Masks, Elevation/Resistance Training Mask and Face Mask/Coverings. Some of these traditional, conventional or modern Respirators, Surgical Masks, Elevation/Resistance Training Mask and Face Mask/Coverings are made and produced by some of the following Companies (e.g., 3M Company, Avon Protection Systems, Inc., Honeywell International, Inc., MSA Safety Inc., Drägerwerk AG & Co. KG, KOWA, Uvex, Halyard Healthcare, McKesson, Molnlycke Health, Hakugen, Shanghai Dasheng, Sinotextiles, Irema and/or Winner Medical).

The removable universal cool filter ice pack insert accessory 400 top surface 403 and a bottom surface 404 is comprised of any of the following material, substance or compounds; High-Density Polyethylene (HDPE), Low-Density Polyethylene (LDPE), and/or materials that is non-toxic or harmful to the human's skin. The removable universal cool filter ice pack insert accessory 400 sealed pouch 406 is filled with a non-toxic water-soluble gel filling material, substance or compounds used for (e.g., gel packs, instant cold packs, clay packs, PCM packs). The non-toxic water-soluble gel filling material, substance or compounds stored in the removable universal cool filter ice pack insert accessory 400 sealed pouch 406 can be but not limited to being comprised of a slow-flowing non-toxic materials, substances or compounds that may include but not limited to the following; Phase Change Material (PCM), hydroxyethyl cellulose, sodium polyacrylate, vinyl-coated silica gel, water, amylose and/or amylopectin. The removable universal cool filter ice pack insert accessory 400 can be permanent affixed, stored, housed, secured or placed within a traditional, conventional or modern respirator, surgical mask or mask recommended by respirator standards in various countries throughout the world such as but not limited to the following: N95/N99—United States NIOSH, FFP2/FFP3—Europe CE EN 149:2001+A1:2009, KN95—China GB2626-2006, Special 1st—Korea KMOEL—2017-64, P95—Mexico NOM-116-2009, P2/P3—Australia AS/NZS 1716:2012, DS/DL2/DL3—Japan JMHLW-200. (e.g., disposable respirators, half-face respirators or full-face respirators).

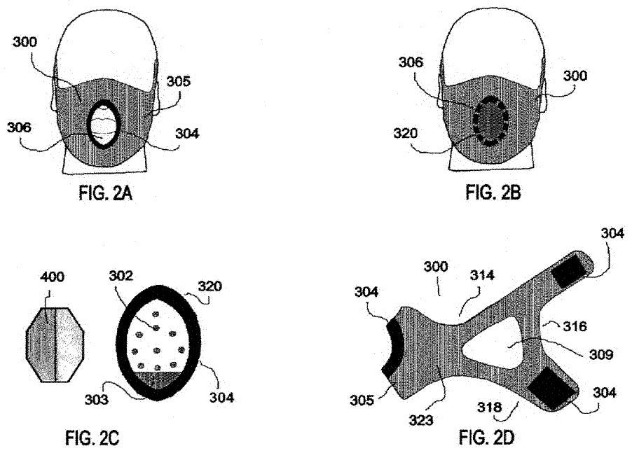

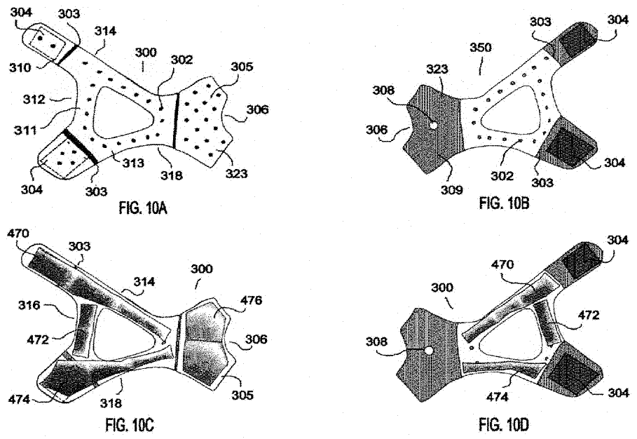

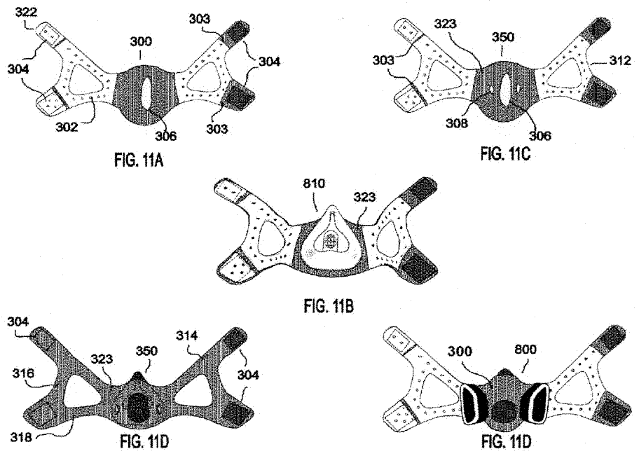

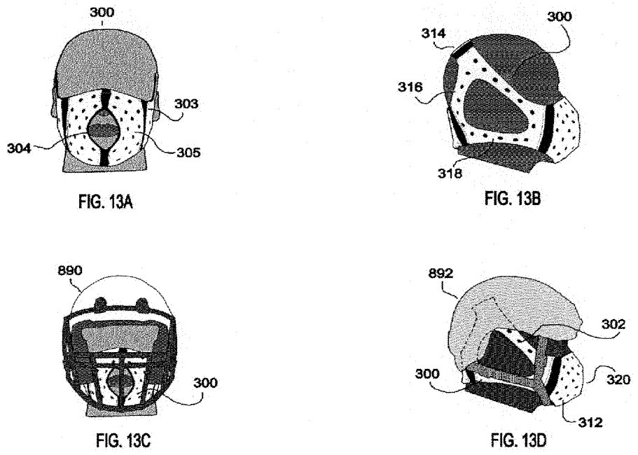

Referring now to the drawings FIGS. 2A-2D, 4A-4B, 5D-5F, 10A-10D, 11A-11E, 12A-12E and 13A-13F. The multifunctional face mask assembly 100 comprising the universal cool release vent harness sleeve/strap accessory 300. The universal cool release vent harness sleeve/strap accessory 300 generally configured to optionally receive, house or secure a respirator, Nebulizer mask or nose cup comprising a Filtration Mask as seen in FIG. 12A, a handheld/portable Nebulizer as seen in FIG. 12B and elastomeric half facepiece reusable respirator seen in FIG. 12C. The universal cool release vent harness sleeve/strap accessory comprises two identical or equal sides, a left side 322 and a right side 324 coupled or joined to form an oval nose cup shaped cavity opening 306 as seen in FIG. 2A around the nose and mouth area also seen in FIGS. 11A, 11C and 13A. Both left side 322 and right side 324 have a planar flat outer surface 323 as seen in FIGS. 2D and 11D and a top side 325, a bottom side 326, a front side 327, a back side 328 as seen in FIGS. 10A and 10B.

As seen in FIGS. 2B and 2C the universal cool release vent harness sleeve/strap accessory 300 separable fasteners 304 around its oval nose cup shaped cavity opening 306 configured to hold, secure or fasten a filter cover pouch 320 seen in FIG. 2C. The filter cover pouch 320 allows placement of a removable universal cool filter ice pack insert accessory 400 within its sleeve/strap pouch opening 303. The filter cover pouch 320 seen in FIG. 2C is secured, attached or fastened by means of the separable fasteners 304 over the oval nose cup shaped cavity opening 306 seen in FIG. 2B allowing the universal cool release vent harness sleeve/strap accessory 300 to be used or function as a filtration mask providing optional cooling in front of the nose and mouth as seen in FIG. 2B. As seen in FIG. 13C-13D, shows the universal cool release vent harness sleeve/strap accessory 300 being used as a football helmet face covering 890 filtration mask on its reverse side revealing the cool release vents 302 cavity opening seen in FIG. 13C without the filter cover pouch 320 being fastened seen in FIGS. 2B-2C. Seen in FIG. 13D shows the universal cool release vent harness sleeve/strap accessory 300 being used as a tactical helmet face covering 892 filtration mask on its reverse side revealing the cool release vents 302 cavity opening with the filter cover pouch 320 fastened by means of separable fasteners 304. The planar surface of the sleeve strap pouch insert 312 comprising cool release vents 302 cavity opening to allow a faster cooling released sensation to the face, head and neck from the removable universal cool filter ice pack insert accessory 400 housed or secured within the sleeve strap pouch insert 312 to provide cool ambient microclimate around the face and aid in cooling the regulation of cerebral blood flow by means of the Hypothalamus which is responsible for regulating body temperature. The planar surface of the sleeve strap pouch insert 312 comprising an absorbent moisture-wicking and breathable material used to aid in absorbing droplets and sweat as seen in FIGS. 10A and 10B. As seen in FIG. 10A, the two identical or equal sides a left side 322 and a right side 324 have a flat planar inner surface 323 comprising a universal cool release vent harness sleeve/strap accessory pouch insert 312 tracking both identical or equal sides a left side 322 and a right side 324 to receive, house or store the removable universal cool filter ice pack insert accessory 400 within its sleeve/strap pouch insert opening 303. The sleeve/strap pouch insert 312 comprising a sleeve/strap face pouch 305, a sleeve/strap upper pouch 310, a sleeve/strap middle pouch 311, a sleeve/strap lower pouch 313 as seen in FIG. 10A. Seen in FIG. 10C shows tracking placement of removable universal cool filter ice pack insert accessory 400 comprising a upper ice pack 470 tracking from the face and temples to the top of the head, a middle ice pack 472 tracking up and down on the back side of the head, a lower ice pack 474 tracking from the face and lower neck to the back of the nape of the neck, a face area ice pack 476 tracking from the nose to the chin.

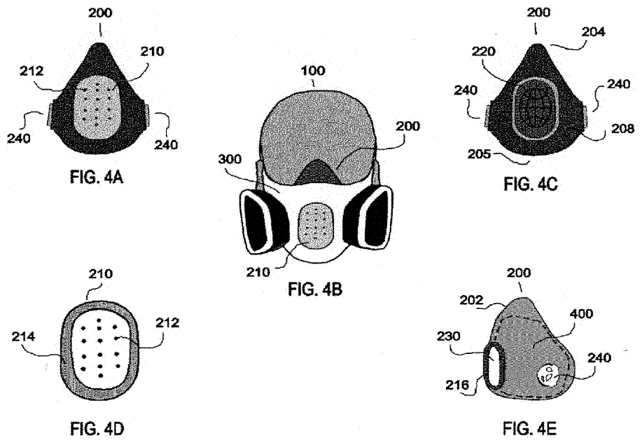

As seen in FIGS. 4A-4B, 11D, 12E and 13B The universal cool release vent harness sleeve/strap accessory 300 is configured or constructed comprising a upper strap 314 contours or tracks around the head above the ears joined by separable fasteners 304 seen in FIG. 12E and 13B, a middle strap 316 contours or tracks around the back of the head behind the ears seen in FIG. 12E and 13B, a lower strap 318 contours or tracks to the back of the neck below the ears joined by separable fasteners 304 seen in FIG. 12E and 13B. The upper strap 314 and lower strap 318 extends from the rear end of the face pouch insert 305 area as seen in FIG. 2D. The middle strap 316 is coupled or joined to the upper strap 314 and lower strap 318 as seen in FIG. 2D. The face pouch insert 305 area, the upper strap 314, the middle strap 316 and the lower strap 318 forms or comprises a triangle cavity opening 309 to allow placement over the ears. As seen in FIGS. 10B, 11C-E, 12B and 12C the universal cool release vent harness sleeve/strap accessory 300 is also shaped, constructed or configured in the form of universal cool release vent respirator harness sleeve/strap accessory 350 as best seen in FIG. 11C, 12B and 12C to receive a respirator, Nebulizer or mask nose cup (e.g., half facepiece elastomeric reusable respirator nose cup, handheld/portable Nebulizer mask nose cup etc.) comprising a cartridge filter adapter valve opening 308 to receive and secure a mask comprising a cartridge filter 352 seen in FIG. 12C or exhalation valve opening 860 seen in FIG. 12B. As seen in FIG. 10B the universal cool release vent respirator harness sleeve/strap accessory 350 comprising a flat planar surface 323 with a cavity opening 309 located by the nose and mouth area as best seen in FIGS. 10B and 11C. As seen in FIG. 4B and 11D the universal cool release vent respirator harness sleeve/strap accessory 350 constructed, configured, formed and contoured to house a Filtrator™ nose cup accessory 200.

The universal cool release vent harness sleeve/strap accessory 300 can be shaped, constructed or configured into a plurality of shapes or forms comprising such as without limitations to be made of any elastic or non-elastic material or fabric constructions. The universal cool release vent harness sleeve/strap accessory 300 can be but not limited to being constructed from a plurality type of breathable materials or composites. These materials or composites can be but not limited to being comprised primarily of; Polyvinyl Acetate (PVA) absorbent material to provide a cool sensation to the skin of the wearer. Polyvinyl Acetate (PVA) absorbent material also has the ability to aid the removable universal cool filter ice pack insert accessory reduce the moisture, heat and humidity. Nanofiber composite material using NanoSilver Technology. The universal cool release vent harness sleeve/strap accessory 300 can be but not limited to include or be constructed using any of the following materials that is non-toxic or harmful to the human's skin or respiratory system; Cotton, Jersey Box Lining Fabric, Polyester Sandwich Spacer Mesh, Polyester Warp Knitting Sandwich Mesh, Microfiber Cooling Material, SMS Polypropylene fabric, Polyester (PES), Heat-Activated Thermoplastics that includes ethylene vinyl acetate (EVA), Neoprene, Breathable nylon mesh, Microfiber Material, polyolefin materials, Poly-Nylon, Spandex, N95-rated material, smart fabrics, E-fabrics (electronic fabrics), Chamois, Foam, Halyard Health Sequential Sterilization Fabrics Interfacing, Aluminum Coated PET film, High-Density Polyethylene (HDPE), Low-Density Polyethylene (LDPE) and/or Antimicrobial Fabrics.

Referring to drawings FIGS. 4A-4E, 10B, 10D and 11D. The multifunctional face mask assembly 100 comprising the Filtrator™ nose cup accessory 200. The Filtrator™ nose cup accessory 200 generally configured to optionally receive or secure a cartridge filter 352 seen in FIG. 4B by means of the cartridge filter adapter 240 seen in FIG. 4E. The Filtrator™ nose cup accessory 200 comprises a body cavity or opening 202 contoured to interchangeably house, secure a removable universal cool filter ice pack insert accessory 400 nested in its housing embodiment as seen in FIG. 4E. The Filtrator™ nose cup accessory 200 have a top side 204, a bottom side 205, a left side 206, a right side 207 and a front side 208 as seen in FIG. 4C. The Filtrator™ nose cup accessory 200 allows the removable universal cool filter ice pack insert accessory 400 to provide a cool ambient microclimate around the face aiding to reduce humidity, moisture, heat and carbon dioxide levels. The Filtrator™ nose cup accessory 200 also comprising a detachable filter cover 210 optionally attached or fastened to its embodiment by means of clamping, fastening or securing the filter cover wall 214 to the filter cover wall clamp 216 as seen in FIGS. 4D and 4E. The detachable filter cover 210 comprises a plurality of filter cover air vents 212 cavity openings to allow filtered ventilation to be released from the Filtrator™ nose cup accessory 200 housing or microclimate by means of the filter 230 as seen in FIGS. 4B, 4D and 4E. The Filtrator™ nose cup accessory 200 also comprises a filter bracket holder with filtration vents 220 to hold, support and secure a filter 230 as seen in FIGS. 4C and 4E. The Filtrator™ nose cup accessory 200 is optionally housed, secured, or attached to the multifunctional face mask assembly 100 by means of the universal cool release vent respirator harness sleeve/strap accessory 350.

The Filtrator™ nose cup accessory 200 embodiment can be shaped, constructed or configured into a plurality of shapes or forms comprising such as without limitations to be made of any silicone based material, elastic or non-elastic material or constructions. The Filtrator™ nose cup accessory 200 can be but not limited to include or be constructed using any of the following materials that is non-toxic or harmful to the human's skin or respiratory system.

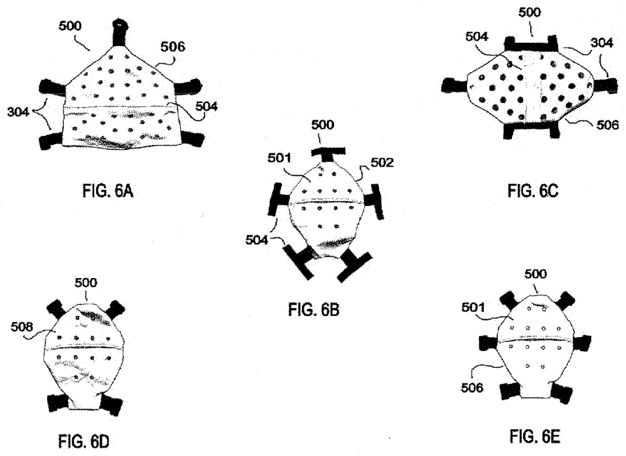

Referring to drawings FIGS. 1A-1B, 1D-1E, 4E, 6A-6E, 7A-7E, 8A-8F and 9A-9F. The multifunctional face mask assembly 100 comprising the universal cool filter ice pack insert pouch accessory 500 comprising a plurality of cool release vents 302 cavity opening. The universal cool filter ice pack insert pouch accessory 500 generally have a top surface 501 and a bottom surface 502 as seen in FIGS. 1B, 1E, 6A-6F. The top surface 501 and the bottom surface 502 are joined by their perimeter edges 506 as best seen in FIG. 1E to form a pocket housing compartment 508 cavity seen in FIG. 6A to receive a removable universal cool filter ice pack insert accessory 400. The top surface 501 comprises a planar surface with a plurality of cool release vents 302 cavity opening as best seen in FIGS. 1B and 1E. As seen in FIGS. 6A-6E the top surface 501 also comprises a pocket flap opening 504 to allow the universal cool filter ice pack insert pouch accessory 500 to be housed within its pocket housing compartment 508. The bottom surface 502 comprises a planar flat surface. The universal cool filter ice pack insert pouch accessory 500 also comprises absorbent moisture-wicking and breathable material used to aid in absorbing droplets and sweat. The universal cool filter ice pack insert pouch accessory 500 cool release vents 302 cavity opening provide a faster cool released sensation to aid in a cool ambient microclimate around the face being housed in the Filtrator™ nose cup accessory 200 as seen in FIG. 4E or back of the head as seen in FIGS. 8A and 8B to aid in lowering skin temperature around the face and/or head by means of the removable universal cool filter ice pack insert accessory 400. The universal cool filter ice pack insert pouch accessory 500 comprises a plurality of separable fasteners 304 affixed or joined around its perimeter edges 506 as seen in FIGS. 6A-6E. The separable fasteners 304 allows the universal cool filter ice pack insert pouch accessory 500 to be fastened, joined or attached to traditional, conventional or modern Respirators, Surgical Masks, Elevation/Resistance Training Mask and Face Mask/Coverings head harness 802 as seen in FIGS. 8A-8B and 8D and 8E. FIG. 8A shows the universal cool filter ice pack insert pouch accessory 500 within its pocket housing compartment 508 interchangeably attached, fastened or secured by means of separable fasteners 304 being contoured around the back of the head to the head harness 802 of a 3M Elastomeric Half Facepiece Reusable Respirator 800. Seen in FIG. 8B shows the universal cool filter ice pack insert pouch accessory 500 revealing the cool release vents 302 cavity opening, separable fasteners 304, pocket flap opening 504 and removable universal cool filter ice pack insert accessory 400 housed and secured in its pocket housing compartment 508. FIG. 8D shows the housing of the universal cool filter ice pack insert pouch accessory 500 interchangeably attached, fastened or secured by means of separable fasteners 304 being contoured around the back of the head to the head harness 802 of a 3M full facepiece respirator 830.

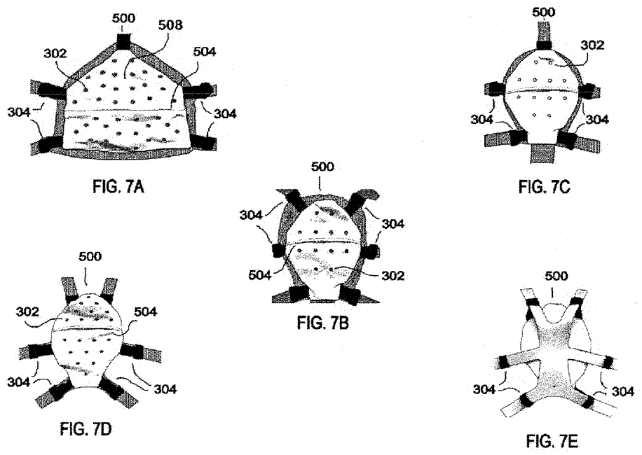

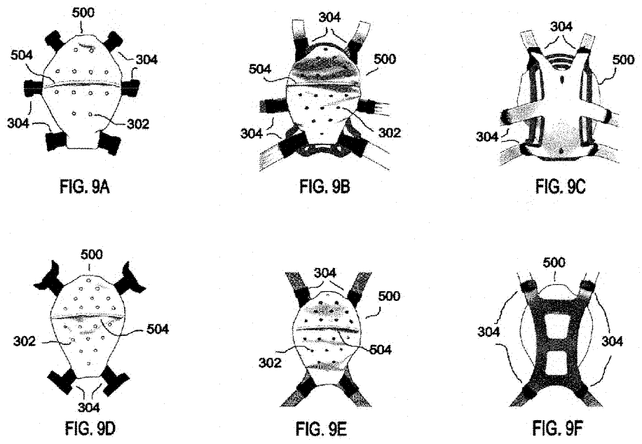

As seen in FIGS. 7A-7E, 9B, 9C, 9E and 9F. The universal cool filter ice pack insert pouch accessory 500 comprises a plurality of optionally separable fasteners 304 to fasten, secure, attach or hold the universal cool filter ice pack insert pouch accessory 500 to a plurality of head harness 802 constructed or configured for traditional, conventional or modern Respirators, Surgical Masks, Elevation/Resistance Training Mask and Face Mask/Coverings. FIG. 7A shows the universal cool filter ice pack insert pouch accessory 500 fastened, secured and attached to a MSA Ultra Elite Full-Facepiece Respirator Head Harness by means of separable fasteners 304 revealing cool release vents 302 cavity opening, pocket flap opening 504 and pocket housing compartment 508. FIG. 7B shows the universal cool filter ice pack insert pouch accessory 500 fastened, secured and attached to an Avon M50/FM50 CBRN Gas Mask Head Harness by means of separable fasteners 304 revealing cool release vents 302 cavity opening, pocket flap opening 504 and pocket housing compartment 508. FIG. 7C shows the universal cool filter ice pack insert pouch accessory 500 fastened, secured and attached to a Scott AV-3000 SCBA Head Harness by means of separable fasteners 304 revealing cool release vents 302 cavity opening, pocket flap opening 504 and pocket housing compartment 508. FIG. 7D shows the universal cool filter ice pack insert pouch accessory 500 fastened, secured and attached to a 3M™ Head Harness FF-400-04 by means of separable fasteners 304 revealing cool release vents 302 cavity opening, pocket flap opening 504 and pocket housing compartment 508. FIG. 7E shows back side of the universal cool filter ice pack insert pouch accessory 500 fastened, secured and attached to a 3M™ Head Harness FF-400-04 by means of separable fasteners 304. FIG. 9B shows the universal cool filter ice pack insert pouch accessory 500 fastened, secured and attached to a 3M™ Comfort Cradle Head Harness Attachment FF-400-06 by means of separable fasteners 304 revealing cool release vents 302 cavity opening, pocket flap opening 504 and pocket housing compartment 508. FIG. 9C shows back side of the universal cool filter ice pack insert pouch accessory 500 fastened, secured and attached to a 3M™ Comfort Cradle Head Harness Attachment FF-400-06 by means of separable fasteners 304. FIG. 9E shows the universal cool filter ice pack insert pouch accessory 500 fastened, secured and attached to a 3M Full Facepiece Respirator 6000 Series Head Harness by means of separable fasteners 304 revealing cool release vents 302 cavity opening, pocket flap opening 504 and pocket housing compartment 508. FIG. 9F shows back side of the universal cool filter ice pack insert pouch accessory 500 fastened, secured and attached to a 3M Full Facepiece Respirator 6000 Series Head Harness by means of separable fasteners 304.

[symbol]

The universal cool filter ice pack insert pouch accessory 500 is constructed, configured or shaped in a plurality of forms some comprising odd shapes for placement within the multifunctional face mask assembly embodiment 100 Filtrator™ nose cup accessory 200 and/or universal placement in traditional, conventional or modern Respirators, Surgical Masks, Silicone Based Mask and Face Mask/Coverings made constructed or manufacture by (e.g., 3M Company, Avon Protection Systems, Inc., Honeywell International, Inc., MSA Safety Inc., Drägerwerk AG & Co. KG, KOWA, Uvex, Halyard Healthcare, McKesson, Molnlycke Health, Hakugen, Shanghai Dasheng, Sinotextiles, Irema and/or Winner Medical).

The universal cool filter ice pack insert pouch accessory 500 can be made of any absorbent wicking material, elastic or non-elastic material or fabric compositions. The universal cool filter ice pack insert pouch accessory 500 can be but not limited to being constructed from a plurality type of breathable materials or composites. These materials or composites can be but not limited to being comprised primarily of; Polyvinyl Acetate (PVA) absorbent material to provide a cool sensation to the skin of the wearer. Polyvinyl Acetate (PVA) absorbent material also has the ability to aid the removable universal cool filter ice pack insert accessory reduce the moisture, heat and humidity. Nanofiber composite material using NanoSilver Technology. The universal cool filter ice pack insert pouch accessory 500 can be but not limited to include or be constructed using any of the following materials that is non-toxic or harmful to the human's skin or respiratory system; Cotton, Jersey Box Lining Fabric, Polyester Sandwich Spacer Mesh, Polyester Warp Knitting Sandwich Mesh, Microfiber Cooling Material, SMS Polypropylene fabric, Polyester (PES), Heat-Activated Thermoplastics that includes ethylene vinyl acetate (EVA), Neoprene, Breathable nylon mesh, Microfiber Material, polyolefin materials, Poly-Nylon, Spandex, N95-rated material, smart fabrics, E-fabrics (electronic fabrics), Chamois, Foam, Halyard Health Sequential Sterilization Fabrics Interfacing, Aluminum Coated PET film, High-Density Polyethylene (HDPE), Low-Density Polyethylene (LDPE) and/or Antimicrobial Fabrics.

It will be understood by those skilled in the art that the present embodiments extend beyond the disclosed embodiments to other alternative embodiments and/or uses of the embodiments and obvious modifications and equivalents thereof. The various features and embodiments disclosed herein are for purposes of illustration and are not intended to be limiting, without departing from the true scope and spirit being indicated by the following claims presented. In addition, while several variations of the embodiments have been shown and described in detail, other modifications, which are within the scope of these embodiments, will be readily apparent to those of skill in the art based upon this disclosure. It is also anticipated that various combination of the specific features and parts of the embodiments may be made and still fall within the scope of the invention. It should be understood that various features and parts of the disclosed embodiments can be combined with or substituted for one another in order to form varying modes of the disclosed embodiments. Thus, it is intended that the scope of at least some of the present technology and embodiments herein disclosed should not be limited by the particular disclosed embodiments described above.

What is claimed is:

1. An apparatus comprising:
a multifunctional face mask assembly embodiment comprising:
a nose cup accessory, the nose cup accessory including an opening into the cavity, a top side, a bottom side, a left side, a right side, front side configured or contoured to receive a filter cover and to interchangeably to receive and house a removable universal cool filter ice pack insert accessory to be nested in its opening cavity;
a removable universal cool filter ice pack insert accessory having a top surface, a bottom surface coupled by their perimeter edges constructed or configured into a plurality of shapes or forms and heat-sealed lines to form sealed pouches filled with non-toxic substance or compounds;
a universal cool release vent harness sleeve/strap accessory having two identical or equal sides a left side and a right side coupled or joined to form an oval nose cup shaped cavity opening, both left side and right side have a planar flat outer surface and a top side, a bottom side, a front side and a back side, having separable fasteners, a upper strap contours or tracks around the head above the ears joined by separable fasteners, a middle strap contours or tracks around the back of the head behind the ears, a lower strap contours or tracks to the back of the neck below the ears joined by separable fasteners, the upper strap and lower strap extend from the rear end of the face pouch insert area, the middle strap is coupled, combined or joined to the upper strap and lower strap to form or comprise a triangle cavity opening to allow placement over the ears, also having a flat planar inner surface having a universal cool release vent harness sleeve/strap accessory pouch insert tracking to receive house or store a removable universal cool filter ice pack insert accessory within its sleeve/strap pouch insert opening, also having cool release vents cavity opening and comprising absorbent moisture-wicking and breathable material used to aid in absorbing droplets and sweat;
a universal cool release vent respirator harness sleeve/strap accessory having a flat planar surface with a cavity opening located by the nose and mouth area constructed configured, formed and contoured to house and receive a respirator, Nebulizer or mask nose cup accessory;
a universal cool filter ice pack insert pouch accessory having a top surface, a bottom surface joined by their perimeter edges to form a pocket housing compartment cavity to receive a removable universal cool filter ice pack insert accessory, the top surface having a planar surface with a plurality of cool release vents cavity opening, a top surface having a pocket flap opening to allow the universal cool filter ice pack insert pouch accessory to be housed within its pocket housing compartment, a bottom surface comprises a planar flat surface, also comprises absorbent moisture-wicking and breathable material used to aid in absorbing droplets and sweat and having a plurality of separable fasteners affixed or joined around its perimeter edges to allow the universal cool filter ice pack insert pouch accessory to be fastened, joined or attached to traditional, conventional or modern Respirators, Surgical Masks, Elevation/Resistance Training Mask and Face Mask/Coverings head harness.

2. The apparatus of claim 1, wherein the nose cup accessory comprises a cavity opening to interchangeably to receive and house a removable universal cool filter ice pack insert accessory to be nested in its housing embodiment.

3. The apparatus of claim 1, wherein the nose cup accessory is optionally housed, secured, or attached to the multifunctional face mask assembly by means of the universal cool release vent respirator harness sleeve/strap accessory

4. The apparatus of claim 1, wherein the nose cup accessory generally configured to optionally receive or secure a cartridge filter by means of the cartridge filter adapters.

5. The apparatus of claim 1, wherein the nose cup accessory filter cover comprises a plurality of filter cover air vents cavity openings to allow filtered ventilation to be released from the nose cup accessory.

6. The apparatus of claim 1, wherein the removable universal cool filter ice pack insert accessory comprises heat sealed lines configured to allow pliable bending to be optionally nested within the universal cool release vent harness sleeve/strap accessory sleeve strap pouch insert, nose cup housing of a respirator, elevation/resistance training mask, and face mask/covering.

7. The apparatus of claim 1, wherein the removable universal cool filter ice pack insert accessory optionally housed or secured in the universal cool release vent harness sleeve/strap accessory covering, tracking or crossing the internal carotid artery and external carotid arteries providing aid in lowering skin temperature around the face, head and neck to aid in cooling the regulation of cerebral blood flow by means of the Hypothalamus which is responsible for regulating body temperature.

8. The apparatus of claim 1, wherein the removable universal cool filter ice pack insert accessory comprising a upper ice pack tracking from the face and temples to the top of the head, a middle ice pack tracking up and down on the back side of the head, a lower ice pack tracking from the face and lower neck to the back of the nape of the neck, a face area ice pack tracking from the nose to the chin.

9. The apparatus of claim 1, wherein the removable universal cool filter ice pack insert accessory may be shaped, constructed or configured into a plurality of shapes or forms comprising such as without limitation; a rectangular, square, circle, heart, clover, star, diamond, oval, octagon, trapezoid, pentagon, hexagon, triangle, parallelogram, rhombus or unique shape that is conformed, adaptable, fashioned, designed or shaped for placement within traditional, conventional or modern Respirators, Surgical Masks, Elevation/Resistance Training Mask and Face Mask/Coverings.

10. The apparatus of claim 1, wherein the removable universal cool filter ice pack insert accessory sealed pouch can be but not limited to being comprised of a non-toxic water-soluble gel filling material, substance or compounds stored in the slow-flowing non-toxic materials, substances or compounds that may include but not limited to the following; Phase Change Material (PCM), hydroxyethyl cellulose, sodium polyacrylate, vinyl-coated silica gel, water, amylose and/or amylopectin.

11. The apparatus of claim 1, wherein the universal cool release vent harness sleeve/strap accessory comprises separable fasteners around its oval nose cup shaped cavity opening configured to hold, secure or fasten a filter cover pouch allowing placement of a removable universal cool filter ice pack insert accessory within its sleeve/strap pouch opening.

12. The apparatus of claim 1, wherein the universal cool release vent harness sleeve/strap accessory having a planar surface of the sleeve strap pouch insert comprising cool release vents cavity opening to allow a faster cooling released sensation to the face, head and neck from the removable universal cool filter ice pack insert accessory housed or secured within the sleeve strap pouch insert to provide cool ambient microclimate around the face and aid in cooling the regulation of cerebral blood flow by means of the Hypothalamus which is responsible for regulating body temperature.

13. The apparatus of claim 1, wherein the universal cool release vent harness sleeve/strap accessory comprising a sleeve/strap face pouch, a sleeve/strap upper pouch, a sleeve/strap middle pouch, a sleeve/strap lower pouch to house the removable universal cool filter ice pack insert accessory.

14. The apparatus of claim 1, wherein the universal cool release vent harness sleeve/strap accessory is also shaped, constructed or configured in the form of universal cool release vent respirator harness sleeve/strap accessory to receive a respirator, Nebulizer or mask nose cup (e.g., half facepiece elastomeric reusable respirator nose cup, handheld/portable Nebulizer mask nose cup etc.) comprising a cartridge filter adapter valve opening to receive and secure a mask comprising a cartridge filter or exhalation valve opening.

15. The apparatus of claim 1, wherein the universal cool release vent harness sleeve/strap accessory can be shaped, constructed or configured into a plurality of shapes or forms comprising such as without limitations to be made of any elastic or non-elastic material or fabric constructions from a plurality type of breathable materials or composites. These materials or composites can be but not limited to being comprised primarily of; Polyvinyl Acetate (PVA) absorbent material to provide a cool sensation to the skin of the wearer and non-toxic or harmful to the human's skin or respiratory system; absorbent material also has the ability to aid the removable universal cool filter ice pack insert accessory reduce the moisture, heat and humidity. Nanofiber composite material, Cotton, Jersey Box Lining Fabric, Polyester Sandwich Spacer Mesh, Polyester Warp Knitting Sandwich Mesh, Microfiber Cooling Material, SMS, Polyester (PES), Heat-Activated Thermoplastics that includes ethylene vinyl acetate (EVA), Neoprene, Breathable nylon mesh, Microfiber Material, polyolefin materials, Poly-Nylon, Spandex, N95-rated material, smart fabrics, E-fabrics (electronic fabrics), Chamois, Foam, Aluminum Coated PET film and/or Antimicrobial Fabrics.

16. The apparatus of claim 1, wherein the universal cool filter ice pack insert pouch accessory having separable fasteners allows the universal cool filter ice pack insert pouch accessory to be fastened, joined or attached to traditional, conventional or modern Respirators, Surgical Masks, Elevation/Resistance Training Mask and Face Mask/Coverings head harness.

17. The apparatus of claim 1, wherein the universal cool filter ice pack insert pouch accessory allows the universal cool filter ice pack insert pouch accessory interchangeably attached, fastened or secured by means of separable fasteners being contoured around the back of the head to the head harness of a full facepiece respirator

18. The apparatus of claim 1, wherein the universal cool filter ice pack insert pouch accessory comprises a plurality of optionally separable fasteners to fasten, secure, attach or hold the universal cool filter ice pack insert pouch accessory to a plurality of head harness constructed or configured for traditional, conventional or modern Respirators, Surgical Masks, Elevation/Resistance Training Mask and Face Mask/Coverings.

19. The apparatus of claim 1, wherein the universal cool filter ice pack insert pouch accessory is constructed, configured or shaped in a plurality of forms some comprising odd shapes for placement within the multifunctional face mask assembly Filtrator™ nose cup accessory and/or universal placement in traditional, conventional or modern Respirators, Surgical Masks, Silicone Based Mask and Face Mask/Coverings made constructed or manufacture by (e.g., 3M Company, Avon Protection Systems, Inc., Honeywell International, Inc., MSA Safety Inc., Drägerwerk AG & Co. KG, KOWA, Uvex, Halyard Healthcare, McKesson, Molnlycke Health, Hakugen, Shanghai Dasheng, Sinotextiles, Irema and/or Winner Medical).

20. The apparatus of claim 1, wherein the universal cool filter ice pack insert pouch accessory can be made of any absorbent wicking material, elastic or non-elastic material or fabric compositions.
